(19) United States
(12) Patent Application Publication
(10) Pub. No.: US 2023/0293258 A1
(43) Pub. Date: Sep. 21, 2023

(54) MEDICAL ARM CONTROL SYSTEM, MEDICAL ARM CONTROL METHOD, AND PROGRAM

(71) Applicant: SONY GROUP CORPORATION, TOKYO (JP)

(72) Inventor: NAOKI NISHIMURA, TOKYO (JP)

(21) Appl. No.: 18/005,064

(22) PCT Filed: Jun. 28, 2021
(86) PCT No.: PCT/JP2021/024287

§ 371 Date: Jan. 11, 2023

(30) Foreign Application Priority Data
Jul. 20, 2020 (JP) 2020-124035

(51) Int. Cl. A61B 90/00; A61B 1/00

(52) U.S. Cl. CPC A61B 90/361; A61B 1/00009; A61B 1/00188

(57) ABSTRACT

Provided is a medical arm control system (200) including: a range setting unit (216) that sets a cutout range in which a second image is cut out from a first image by a medical observation device supported by an arm unit; a light amount acquisition unit (224) that acquires a light amount distribution of the first image and a light amount distribution of the second image; a distance acquisition unit (222) that acquires distance information indicating a distance between a subject of the medical observation device and the medical observation device; and an arm control unit (203) that controls the arm unit on the basis of the light amount distributions of the first and second images and the distance information.

### FIELD

The present disclosure relates to a medical arm control system, a medical arm control method, and a program.

### BACKGROUND

In recent years, in endoscopic surgery, a patient’s abdominal cavity is imaged using an endoscope, and an operator performs surgery while confirming a captured image imaged by the endoscope on a display. For example, Patent Literature 1 and Patent Literature 2 below disclose a technique for suitably adjusting imaging conditions of an endoscope.

### CITATION LIST

### Patent Literature

Patent Literature 1: JP 2013-144008 A
Patent Literature 2: JP 2013-042998 A

### SUMMARY

### Technical Problem

However, in the above-described technique, there is a limit to further improving visibility of an image obtained by an endoscope or a part of the image desired by an operator. Therefore, the present disclosure proposes a medical arm control system, a medical arm control method, and a program that can make visibility of an image more preferable.

### Solution to Problem

According to the present disclosure, there is provided a medical arm control system including: a range setting unit that sets a cutout range in which a second image is cut out from a first image by a medical observation device supported by an arm unit; a light amount acquisition unit that acquires a light amount distribution of the first image and a light amount distribution of the second image; a distance acquisition unit that acquires distance information indicating a distance between a subject of the medical observation device and the medical observation device; and an arm control unit that controls the arm unit based on the light amount distributions of the first and second images and the distance information.

Furthermore, according to the present disclosure, there is provided a medical arm control method, by a medical arm control device, including: setting a cutout range in which a second image is cut out from a first image by a medical observation device supported by an arm unit; acquiring a light amount distribution of the first image and a light amount distribution of the second image; acquiring distance information indicating a distance between a subject of the medical observation device and the medical observation device; and controlling the arm unit based on the light amount distributions of the first and second images and the distance information.

Furthermore, according to the present disclosure, there is provided a program causing a computer to function as: a range setting unit that sets a cutout range in which a second image is cut out from a first image by a medical observation device supported by an arm unit; a light amount acquisition unit that acquires a light amount distribution of the first image and a light amount distribution of the second image; a distance acquisition unit that acquires distance information indicating a distance between a subject of the medical observation device and the medical observation device; and an arm control unit that controls the arm unit based on the light amount distributions of the first and second images and the distance information.

### BRIEF DESCRIPTION OF DRAWINGS

FIG. 1 is a diagram illustrating an example of a schematic configuration of an endoscopic surgical system to which a technique according to the present disclosure can be applied.

FIG. 2 is a block diagram illustrating an example of a functional configuration of a camera head and a camera control unit (CCU) illustrated in FIG. 1.

FIG. 3 is a schematic diagram illustrating a configuration of a stereo endoscope according to an embodiment of the present disclosure.

FIG. 4 is a block diagram illustrating an example of a configuration of a medical observation system according to the embodiment of the present disclosure.

FIG. 5 is a block diagram illustrating an example of a configuration of a control system 2 according to a first embodiment of the present disclosure.

FIG. 6 is a flowchart of a control method according to the first embodiment of the present disclosure.

FIG. 7 is an explanatory diagram for explaining the first embodiment of the present disclosure.

FIG. 8 is a graph (part 1) for explaining the control method in the first embodiment of the present disclosure.

FIG. 9 is a graph (part 2) for explaining the control method in the first embodiment of the present disclosure.

FIG. 10 is a graph (part 3) for explaining the control method in the first embodiment of the present disclosure.

FIG. 11 is a graph (part 4) for explaining the control method in the first embodiment of the present disclosure.

FIG. 12 is a flowchart of a control method according to a second embodiment of the present disclosure.

FIG. 13 is an explanatory diagram for explaining the second embodiment of the present disclosure.

FIG. 14 is a graph (part 1) for explaining the control method in the second embodiment of the present disclosure.

FIG. 15 is a graph (part 2) for explaining the control method in the second embodiment of the present disclosure.

FIG. 16 is a hardware configuration diagram illustrating an example of a computer that implements functions of a control unit.

### DESCRIPTION OF EMBODIMENTS

Hereinafter, preferred embodiments of the present disclosure will be described in detail with reference to the accompanying drawings. Note that, in the present specification and the drawings, components having substantially the same functional configuration are denoted by the same signs, and redundant description is omitted. Furthermore, in the present specification and the drawings, a plurality of components having substantially the same or similar functional configuration may be distinguished by attaching different alphabets after the same sign. However, in a case where it is not particularly necessary to distinguish each of a plurality of components having substantially the same or similar functional configuration, only the same sign is attached.

Note that the description will be given in the following order.
1. Configuration example of endoscopic surgery system 5000
   1.1 Schematic configuration of endoscopic surgery system 5000
   1.2 Detailed configuration example of support arm device 5027
   1.3 Detailed configuration example of light source device 5043
   1.4 Detailed configuration example of camera head 5005 and CCU 5039
   1.5 Configuration example of endoscope 5001
2. Medical observation system
3. Background to creating embodiments of present disclosure
4. First embodiment
   4.1 Detailed configuration example of control system 2
   4.2 Detailed configuration example of stereo endoscope 100
   4.3 Detailed configuration example of control unit 200
   4.4 Control method
5. Second embodiment
   5.1 Detailed configuration example of control system 2, stereo endoscope 100, and control unit 200
   5.2 Control method
6. Summary
7. Hardware configuration
8. Supplement

### 1. Configuration Example of Endoscopic Surgery system 5000

### 1.1 Schematic Configuration of Endoscopic Surgery System 5000

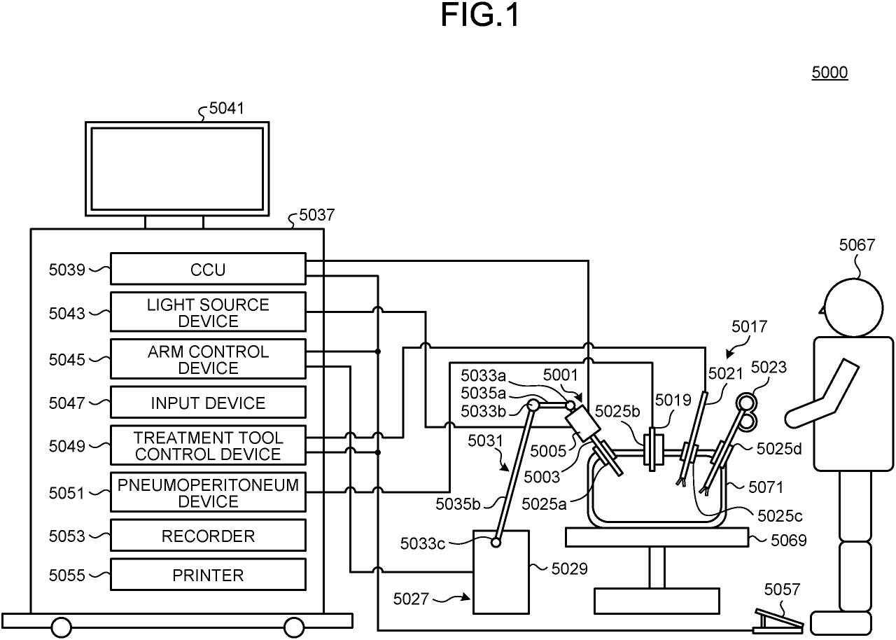

First, before describing details of an embodiment of the present disclosure, a schematic configuration of an endoscopic surgery system 5000 to which a technique according to the present disclosure can be applied will be described with reference to FIG. 1. FIG. 1 is a diagram illustrating an example of a schematic configuration of an endoscopic surgery system 5000 to which a technique according to the present disclosure can be applied. FIG. 1 illustrates a state in which an operator (doctor) 5067 is performing surgery on a patient 5071 on a patient bed 5069 using an endoscopic surgery system 5000. As illustrated in FIG. 1, the endoscopic surgery system 5000 includes an endoscope 5001, other surgical tools 5017, a support arm device 5027 that supports the endoscope 5001, and a cart 5037 on which various devices for endoscopic surgery are mounted. Hereinafter, details of the endoscopic surgery system 5000 will be sequentially described.

### Surgical Tool 5017

In endoscopic surgery, instead of cutting an abdominal wall and opening an abdomen, for example, a plurality of cylindrical piercing instruments called trocars 5025a to 5025d is punctured into the abdominal wall. Then, a lens barrel 5003 of the endoscope 5001 and the other surgical tools 5017 are inserted into a body cavity of the patient 5071 from the trocars 5025a to 5025d. In the example illustrated in FIG. 1, as the other surgical tools 5017, a pneumoperitoneum tube 5019, an energy treatment tool 5021, and forceps 5023 are inserted into the body cavity of the patient 5071. Furthermore, the energy treatment tool 5021 is a treatment tool that performs incision and detachment of tissue, sealing of a blood vessel, or the like by high-frequency current or ultrasonic vibration. However, the surgical tools 5017 illustrated in FIG. 1 is merely an example, and examples of the surgical tools 5017 include various surgical tools generally used in endoscopic surgery, such as tweezers and a retractor.

### Support Arm Device 5027

The support arm device 5027 includes an arm unit 5031 extending from a base part 5029. In the example illustrated in FIG. 1, the arm unit 5031 includes joint parts 5033a, 5033b, and 5033c and links 5035a and 5035b, and is driven by control from an arm control device 5045. Then, the endoscope 5001 is supported by the arm unit 5031, and the position and attitude of the endoscope 5001 are controlled. As a result, stable fixation of the position of the endoscope 5001 can be realized.

### Endoscope 5001

The endoscope 5001 includes the lens barrel 5003 whose region of a predetermined length from a distal end is inserted into the body cavity of the patient 5071, and a camera head 5005 connected to the proximal end of the lens barrel 5003. In the example illustrated in FIG. 1, the endoscope 5001 configured as a so-called rigid scope having a rigid lens barrel 5003 is illustrated, but the endoscope 5001 may be configured as a so-called flexible scope having a flexible lens barrel 5003, and the embodiment of the present disclosure is not particularly limited.

An opening part into which an objective lens is fitted is provided at the distal end of the lens barrel 5003. A light source device 5043 is connected to the endoscope 5001, and light generated by the light source device 5043 is guided to the distal end of the lens barrel by a light guide extending inside the lens barrel 5003, and is emitted toward an observation target in the body cavity of the patient 5071 via the objective lens. Note that, in the embodiment of the present disclosure, the endoscope 5001 may be a front forward-viewing endoscope, an oblique-viewing endoscope, or a side-viewing endoscope, and is not particularly limited.

An optical system and an image sensor are provided inside the camera head 5005, and reflected light (observation light) from the observation target is condensed on the image sensor by the optical system. The observation light is photoelectrically converted by the image sensor, and an electric signal corresponding to the observation light, that is, a pixel signal corresponding to the observation image is generated. The pixel signal is transmitted to a camera control unit (CCU) 5039 as RAW data. Note that the camera head 5005 has a function of adjusting a magnification and a focal length (focus) by appropriately driving the optical system.

Note that, for example, in order to cope with stereoscopic viewing (3D display) or the like, a plurality of the image sensors may be provided in the camera head 5005. In this case, a plurality of relay optical systems are provided inside the lens barrel 5003 in order to guide light to each of the observation fields of view of the plurality of image sensors.

### Various Devices Mounted on Cart

First, under the control of the CCU 5039, a display device 5041 displays an image based on an image signal generated by performing image processing on the pixel signal by the CCU 5039. In a case where the endoscope 5001 is compatible with high-resolution imaging such as 4 K (the number of horizontal pixels 3840 × the number of vertical pixels 2160) or 8 K (the number of horizontal pixels 7680 × the number of vertical pixels 4320), and/or in a case where the endoscope is compatible with 3D display, for example, a display device capable of high-resolution display and/or a display device capable of 3D display corresponding thereto is used as the display device 5041. Furthermore, a plurality of the display devices 5041 having different resolutions and sizes may be provided depending on the application.

Furthermore, an image of a surgical site in the body cavity of the patient 5071 captured by the endoscope 5001 is displayed on the display device 5041. The operator 5067 can perform treatment such as resection of an affected part using the energy treatment tool 5021 and the forceps 5023 while viewing the image of the surgical site displayed on the display device 5041 in real time. Note that, although not illustrated, the pneumoperitoneum tube 5019, the energy treatment tool 5021, and the forceps 5023 may be supported by the operator 5067, an assistant, or the like during surgery.

Furthermore, the CCU 5039 includes a central processing unit (CPU), a graphics processing unit (GPU), and the like, and can integrally control operations of the endoscope 5001 and the display device 5041. Specifically, the CCU 5039 performs, on the pixel signal received from the camera head 5005, various types of image processing for displaying an image based on the pixel signal, such as development processing (demosaic processing), for example. Moreover, the CCU 5039 provides the image signal generated by performing the image processing to the display device 5041. Furthermore, the CCU 5039 transmits a control signal to the camera head 5005 and controls driving thereof. The control signal can include information regarding imaging conditions such as magnification and focal length.

The light source device 5043 includes a light source such as a light emitting diode (LED), for example, and supplies irradiation light for image-capturing a surgical site to the endoscope 5001.

The arm control device 5045 includes, for example, a processor such as a CPU, and operates according to a predetermined program to control driving of the arm unit 5031 of the support arm device 5027 according to a predetermined control method.

An input device 5047 is an input interface for the endoscopic surgery system 5000. The operator 5067 can input various types of information and instructions to the endoscopic surgery system 5000 via the input device 5047. For example, the operator 5067 inputs various types of information regarding surgery, such as physical information of a patient and information regarding a surgical procedure of the surgery, via the input device 5047. Furthermore, for example, the operator 5067 can input an instruction to drive the arm unit 5031, an instruction to change imaging conditions (type, magnification, focal length, and the like of irradiation light) by the endoscope 5001, an instruction to drive the energy treatment tool 5021, and the like via the input device 5047. Note that the type of the input device 5047 is not limited, and the input device 5047 may be various known input devices. As the input device 5047, for example, a mouse, a keyboard, a touch panel, a switch, a foot switch 5057, a lever, and/or the like can be applied. For example, in a case where a touch panel is used as the input device 5047, the touch panel may be provided on a display surface of the display device 5041.

Alternatively, the input device 5047 may be a device worn by the operator 5067, for example, a glasses-type wearable device, a head mounted display (HMD), or the like. In this case, various inputs are performed according to the gesture or the line of sight of the operator 5067 detected by these devices. Furthermore, the input device 5047 can include a camera capable of detecting the movement of the operator 5067, and various inputs may be performed according to the gesture or the line of sight of the operator 5067 detected from an image captured by the camera. Moreover, the input device 5047 can include a microphone capable of collecting the voice of the operator 5067, and various inputs may be performed by the voice via the microphone. As described above, the input device 5047 is configured to be able to input various types of information in a non-contact manner, and thus, in particular, a user (for example, the operator 5067) belonging to a clean area can operate a device belonging to an unclean area in a non-contact manner. Furthermore, since the operator 5067 can operate the instrument without releasing the possessed surgical tool, the convenience of the operator 5067 is improved.

A treatment tool control device 5049 controls driving of the energy treatment tool 5021 for cauterization and incision of tissue, sealing of a blood vessel, or the like. A pneumoperitoneum device 5051 feeds gas into the body cavity of the patient 5071 via the pneumoperitoneum tube 5019 in order to inflate the body cavity for the purpose of securing a visual field by the endoscope 5001 and securing a working space of the operator 5067. A recorder 5053 is a device capable of recording various types of information regarding surgery. A printer 5055 is a device capable of printing various types of information regarding surgery in various formats such as text, image, or graph.

### 1.2 Detailed Configuration Example of Support Arm Device 5027

Moreover, an example of a detailed configuration of the support arm device 5027 will be described. The support arm device 5027 includes the base part 5029 which is a base and the arm unit 5031 extending from the base part 5029. In the example illustrated in FIG. 1, the arm unit 5031 includes the plurality of joint parts 5033a, 5033b, and 5033c and the plurality of links 5035a and 5035b connected by the joint part 5033b, but in FIG. 1, the configuration of the arm unit 5031 is illustrated in a simplified manner for the sake of simplicity. Specifically, the shape, number, and arrangement of the joint parts 5033a to 5033c and the links 5035a and 5035b, the direction of a rotation axis of the joint parts 5033a to 5033c, and the like can be appropriately set so that the arm unit 5031 has a desired degree of freedom. For example, the arm unit 5031 can be suitably configured to have six degrees of freedom or more. As a result, since the endoscope 5001 can be freely moved within a movable range of the arm unit 5031, the lens barrel 5003 of the endoscope 5001 can be inserted into the body cavity of the patient 5071 from a desired direction.

Actuators may be provided in the joint parts 5033a to 5033c, and for example, the joint parts 5033a to 5033c are configured to be rotatable around a predetermined rotation axis by driving of the actuators. The driving of the actuators is controlled by the arm control device 5045, whereby a rotation angle of each of the joint parts 5033a to 5033c is controlled, and the driving of the arm unit 5031 is controlled. As a result, control of the position and attitude of the endoscope 5001 can be realized. At this time, the arm control device 5045 can control the driving of the arm unit 5031 by various known control methods such as force control or position control.

For example, by the operator 5067 appropriately performing an operation input via the input device 5047 (including the foot switch 5057), the driving of the arm unit 5031 may be appropriately controlled by the arm control device 5045 according to the operation input, and the position and attitude of the endoscope 5001 may be controlled. Note that the arm unit 5031 may be operated by a so-called master-slave method. In this case, the arm unit 5031 (slave) can be remotely operated by the operator 5067 via the input device 5047 (master console) installed at a place away from an operating room or in the operating room.

Here, in general, in endoscopic surgery, the endoscope 5001 is supported by a doctor called scopist. On the other hand, in the embodiment of the present disclosure, since the position of the endoscope 5001 can be more reliably fixed without manual operation by using the support arm device 5027, an image of the surgical site can be stably obtained, and surgery can be smoothly performed.

Note that the arm control device 5045 is not necessarily provided in the cart 5037. Furthermore, the arm control device 5045 is not necessarily one device. For example, the arm control device 5045 may be provided in each of the joint parts 5033a to 5033c of the arm unit 5031 of the support arm device 5027, and the drive control of the arm unit 5031 may be realized by a plurality of the arm control devices 5045 cooperating with each other.

### 1.3 Detailed Configuration Example of Light Source Device 5043

Next, an example of a detailed configuration of the light source device 5043 will be described. The light source device 5043 supplies irradiation light when the endoscope 5001 captures an image of a surgical site. The light source device 5043 includes, for example, an LED, a laser light source, or a white light source including a combination thereof. At this time, in a case where a white light source is configured by a combination of RGB laser light sources, the output intensity and the output timing of each color (each wavelength) can be controlled with high accuracy, so that the white balance of the captured image can be adjusted in the light source device 5043. Furthermore, in this case, by irradiating an observation target with the laser light from each of the RGB laser light sources in a time division manner and controlling the driving of the image sensor of the camera head 5005 in synchronization with the irradiation timing, it is also possible to capture an image corresponding to each of RGB in a time division manner. According to this method, a color image can be obtained without providing a color filter in the image sensor.

Furthermore, the driving of the light source device 5043 may be controlled so as to change the intensity of light to be output every predetermined time. By controlling the driving of the image sensor of the camera head 5005 in synchronization with the timing of the change of the light intensity to acquire images in a time division manner and synthesizing the images, it is possible to generate a high dynamic range image without so-called blocked-up shadows and blown-out highlights.

Furthermore, the light source device 5043 may be configured to be able to supply light in a predetermined wavelength band corresponding to special light observation. In the special light observation, for example, so-called narrow band imaging is performed in which a predetermined tissue such as a blood vessel in a mucosal surface layer is imaged with high contrast by irradiating light in a narrower band than irradiation light (that is, white light) at the time of normal observation using wavelength dependency of light absorption in a body tissue. Alternatively, in the special light observation, fluorescence observation for obtaining an image by fluorescence generated by irradiation with excitation light may be performed. In the fluorescence observation, for example, fluorescence from a body tissue can be observed by irradiating the body tissue with excitation light (autofluorescence observation), or a fluorescent image can be obtained by locally injecting a reagent such as indocyanine green (ICG) into a body tissue and irradiating the body tissue with excitation light corresponding to a fluorescence wavelength of the reagent. The light source device 5043 can be configured to be able to supply narrow band light and/or excitation light corresponding to such special light observation.

### 1.4 Detailed Configuration Example of Camera Head 5005 and CCU 5039

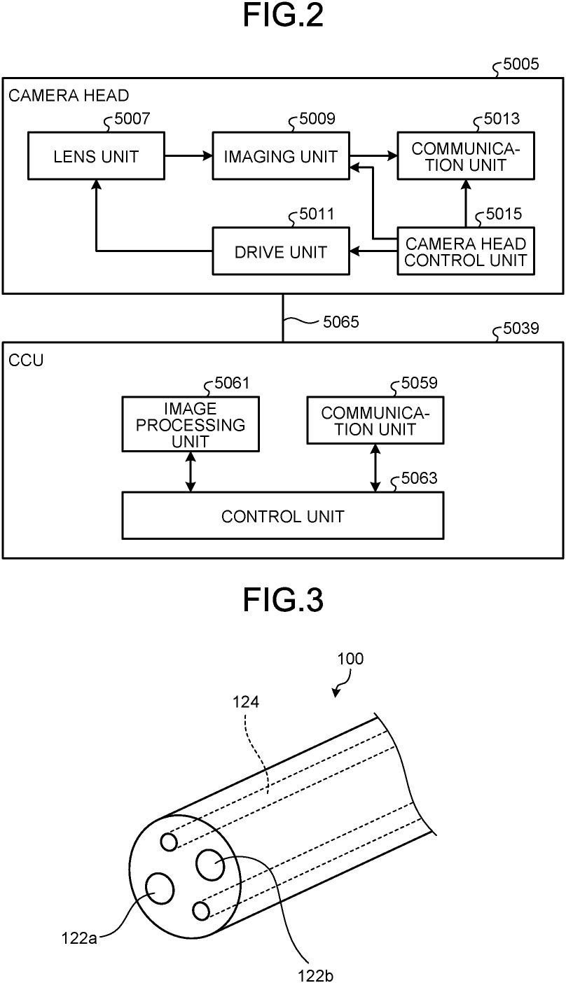

Next, an example of a detailed configuration of the camera head 5005 and the CCU 5039 will be described with reference to FIG. 2. FIG. 2 is a block diagram illustrating an example of functional configurations of the camera head 5005 and the CCU 5039 illustrated in FIG. 1.

Specifically, as illustrated in FIG. 2, the camera head 5005 includes, as functions thereof, a lens unit 5007, an imaging unit 5009, a drive unit 5011, a communication unit 5013, and a camera head control unit 5015. Furthermore, the CCU 5039 includes a communication unit 5059, an image processing unit 5061, and a control unit 5063 as its functions. Then, the camera head 5005 and the CCU 5039 are connected to be bidirectionally communicable by a transmission cable 5065.

First, a functional configuration of the camera head 5005 will be described. The lens unit 5007 is an optical system provided at a connection part with the lens barrel 5003. Observation light taken in from the distal end of the lens barrel 5003 is guided to the camera head 5005 and enters the lens unit 5007. The lens unit 5007 is configured by combining a plurality of lenses including a zoom lens and a focus lens. The optical characteristics of the lens unit 5007 are adjusted so as to condense the observation light on a light receiving surface of an image sensor of the imaging unit 5009. Furthermore, the zoom lens and the focus lens are configured to be movable in position on the optical axis in order to adjust a magnification and a focal point (focus) of a captured image.

The imaging unit 5009 includes the image sensor and is arranged at a subsequent stage of the lens unit 5007. The observation light having passed through the lens unit 5007 is condensed on the light receiving surface of the image sensor, and a pixel signal corresponding to the observation image is generated by photoelectric conversion. A pixel signal generated by the imaging unit 5009 is provided to the communication unit 5013.

As the image sensor constituting the imaging unit 5009, for example, a complementary metal oxide semiconductor (CMOS) type image sensor having a Bayer array and capable of color capturing is used. Note that, as the image sensor, for example, an image sensor that can cope with capturing of a high-resolution image of 4 K or more may be used. By obtaining an image of the surgical site with high resolution, the operator 5067 can grasp the state of the surgical site in more detail, and can progress the surgery more smoothly.

Furthermore, the image sensor included in the imaging unit 5009 may include, for example, a pair of image sensors for acquiring pixel signals for right eye and left eye corresponding to 3D display (stereo endoscope). By performing the 3D display, the operator 5067 can more accurately grasp a depth of a living tissue in the surgical site and grasp a distance to the living tissue. Note that, in a case where the imaging unit 5009 is configured as a multi-plate type, a plurality of the lens units 5007 may be provided corresponding to each image sensor.

Furthermore, the imaging unit 5009 is not necessarily provided in the camera head 5005. For example, the imaging unit 5009 may be provided immediately after an objective lens inside the lens barrel 5003.

The drive unit 5011 includes an actuator, and moves the zoom lens and the focus lens of the lens unit 5007 by a predetermined distance along the optical axis under the control of the camera head control unit 5015. As a result, the magnification and focus of the image captured by the imaging unit 5009 can be appropriately adjusted.

The communication unit 5013 includes a communication device for transmitting and receiving various types of information to and from the CCU 5039. The communication unit 5013 transmits the pixel signal obtained from the imaging unit 5009 as RAW data to the CCU 5039 via the transmission cable 5065. At this time, in order to display the captured image of the surgical site with low latency, the pixel signal is preferably transmitted by optical communication. This is because, at the time of surgery, the operator 5067 performs surgery while observing the state of the affected part with the captured image, and thus, for safer and more reliable surgery, it is required to display a moving image of the surgical site in real time as much as possible. In a case where optical communication is performed, the communication unit 5013 is provided with a photoelectric conversion module that converts an electric signal into an optical signal. The pixel signal is converted into an optical signal by the photoelectric conversion module and then transmitted to the CCU 5039 via the transmission cable 5065.

Furthermore, the communication unit 5013 receives a control signal for controlling driving of the camera head 5005 from the CCU 5039. The control signal includes, for example, information regarding imaging conditions such as information for specifying a frame rate of a captured image, information for specifying an exposure value at the time of imaging, and/or information for specifying a magnification and a focus of a captured image. The communication unit 5013 provides the received control signal to the camera head control unit 5015. Note that the control signal from the CCU 5039 may also be transmitted by optical communication. In this case, the communication unit 5013 is provided with a photoelectric conversion module that converts an optical signal into an electric signal, and the control signal is converted into an electric signal by the photoelectric conversion module and then provided to the camera head control unit 5015.

Note that the imaging conditions such as the frame rate, the exposure value, the magnification, and the focus are automatically set by the control unit 5063 of the CCU 5039 on the basis of the acquired pixel signal. That is, the endoscope 5001 has a so-called auto exposure (AE) function, an auto focus (AF) function, and an auto white balance (AWB) function.

The camera head control unit 5015 controls driving of the camera head 5005 on the basis of the control signal from the CCU 5039 received via the communication unit 5013. For example, the camera head control unit 5015 controls driving of the image sensor of the imaging unit 5009 on the basis of the information to designate the frame rate of the captured image and/or the information to designate the exposure at the time of imaging. Furthermore, for example, the camera head control unit 5015 appropriately moves the zoom lens and the focus lens of the lens unit 5007 via the drive unit 5011 on the basis of the information to designate the enlargement magnification and the focus of the captured image. The camera head control unit 5015 may further have a function of storing information for identifying the lens barrel 5003 and the camera head 5005.

Note that by arranging configurations of the lens unit 5007, the imaging unit 5009, and the like in a sealed structure having high airtightness and waterproofness, the camera head 5005 can have resistance to autoclave sterilization processing.

Next, a functional configuration of the CCU 5039 will be described. The communication unit 5059 includes a communication device for transmitting and receiving various types of information to and from the camera head 5005. The communication unit 5059 receives a pixel signal transmitted from the camera head 5005 via the transmission cable 5065. At this time, as described above, the pixel signal can be suitably transmitted by optical communication. In this case, for optical communication, the communication unit 5059 is provided with a photoelectric conversion module that converts an optical signal into an electrical signal. The communication unit 5059 provides the pixel signal converted into the electric signal to the image processing unit 5061.

Furthermore, the communication unit 5059 transmits a control signal for controlling driving of the camera head 5005 to the camera head 5005. The control signal may also be transmitted by optical communication.

The image processing unit 5061 performs various types of image processing on the pixel signal that is RAW data transmitted from the camera head 5005. Examples of the image processing include various known signal processing such as development processing, high image quality processing (band emphasis processing, super-resolution processing, noise reduction (NR) processing, camera shake correction processing, and/or the like), and/or enlargement processing (electronic zoom processing). Furthermore, the image processing unit 5061 performs detection processing on the pixel signal for performing AE, AF, and AWB.

The image processing unit 5061 includes a processor such as a CPU or a GPU, and the processor operates according to a predetermined program, whereby the above-described image processing and detection processing can be performed. Note that, in a case where the image processing unit 5061 includes a plurality of GPUs, the image processing unit 5061 appropriately divides information related to a pixel signal, and performs image processing in parallel by the plurality of GPUs.

The control unit 5063 performs various types of control related to imaging of the surgical site by the endoscope 5001 and display of the captured image. For example, the control unit 5063 generates a control signal for controlling driving of the camera head 5005. At this time, in a case where the imaging condition is input by the operator 5067, the control unit 5063 generates the control signal on the basis of the input by the operator 5067. Alternatively, in a case where the AE function, the AF function, and the AWB function are mounted on the endoscope 5001, the control unit 5063 appropriately calculates the optimum exposure value, focal length, and white balance according to a result of the detection processing by the image processing unit 5061, and generates the control signal.

Furthermore, the control unit 5063 causes the display device 5041 to display the image of the surgical site on the basis of the image signal generated by performing the image processing by the image processing unit 5061. At this time, the control unit 5063 recognizes various objects in the surgical site image using various image recognition technologies. For example, the control unit 5063 can recognize a surgical tool such as forceps, a specific living body site, bleeding, mist at the time of using the energy treatment tool 5021, and the like by detecting the shape, color, and the like of an edge of an object included in the surgical site image. When displaying the image of the surgical site on the display device 5041, the control unit 5063 superimposes and displays various types of surgery support information on the image of the surgical site using the recognition result. The surgery support information is superimposed and displayed, and presented to the operator 5067, so that the surgery can be more safely and reliably advanced.

The transmission cable 5065 connecting the camera head 5005 and the CCU 5039 is an electric signal cable compatible with electric signal communication, an optical fiber compatible with optical communication, or a composite cable thereof.

Here, in the illustrated example, communication is performed by wire using the transmission cable 5065, but communication between the camera head 5005 and the CCU 5039 may be performed wirelessly. In a case where the communication between the two is performed wirelessly, it is not necessary to lay the transmission cable 5065 in the operating room, so that a situation in which the movement of the medical staff in the operating room is hindered by the transmission cable 5065 can be eliminated.

### <1.5 Configuration Example of Endoscope 5001>

Next, a basic configuration of a stereo endoscope 100 will be described as an example of the endoscope 5001 with reference to FIG. 3. FIG. 3 is a schematic diagram illustrating a configuration of the stereo endoscope 100 according to an embodiment of the present disclosure.

The stereo endoscope 100 is attached to the distal end of the camera head 5005 illustrated in FIG. 1, and the stereo endoscope 100 corresponds to the lens barrel 5003 described in FIG. 1. For example, the stereo endoscope 100 may be rotatable independently of the camera head 4200, for example. In this case, an actuator is provided between the stereo endoscope 100 and the camera head 4200 similarly to the joint parts 5033a, 5033b, and 5033c, and the stereo endoscope 100 rotates with respect to the camera head 4200 by driving of the actuator.

The stereo endoscope 100 is supported by the support arm device 5027. The support arm device 5027 has a function of holding the stereo endoscope 100 instead of the scopist and moving the stereo endoscope so that the stereo endoscope 100 can observe a desired site by the operation of the operator 5067 or the assistant.

Specifically, as illustrated in FIG. 3, the stereo endoscope 100 includes relay lenses 122a and 122b that guide reflected light from the subject to a pair of image sensors (not illustrated) for acquiring right-eye and left-eye pixel signals corresponding to 3D display. Moreover, the stereo endoscope 100 includes a light guide 124 extending inside, and can guide the light generated by the light source device 5043 to a distal end part by the light guide. Furthermore, the light guided by the ride guide may be diffused by a lens (not illustrated). Furthermore, in the embodiment of the present disclosure, a part of a wide-angle image (first image) captured by the endoscope 5001 may be cut out to generate another image (second image) (wide-angle/cutout function). Furthermore, the stereo endoscope 100 can not only obtain an image corresponding to 3D display, but also measure a distance to the subject using a triangulation method using the parallax of pixel signals for the right eye and the left eye.

Note that, in the embodiment of the present disclosure, the endoscope 5001 is not limited to the stereo endoscope 100. In the embodiment of the present disclosure, there is no particular limitation as long as it is an endoscope (wide-angle endoscope) capable of acquiring an image to which a function (wide-angle/cutout function) capable of cutting out a part of the wide-angle image captured by the endoscope 5001 to generate another image can be applied. More specifically, for example, the endoscope 5001 may be a front direct endoscope (not illustrated) that captures the front of the distal end part of the endoscope. Furthermore, for example, the endoscope 5001 may be an oblique endoscope (not illustrated) which has an optical axis having a predetermined angle with respect to a longitudinal axis of the endoscope 5001. Furthermore, for example, the endoscope 5001 may be an endoscope (not illustrated) with a simultaneous imaging function in another direction in which a plurality of camera units having different visual fields is built in the distal end part of the endoscope, and different images can be obtained by the respective cameras.

An example of the endoscopic surgery system 5000 to which the technique according to the present disclosure can be applied has been described above. Note that, here, the endoscopic surgery system 5000 has been described as an example, but the system to which the technique according to the present disclosure can be applied is not limited to such an example. For example, the technique according to the present disclosure may be applied to a microscopic surgery system.

### 2. Medical Observation System

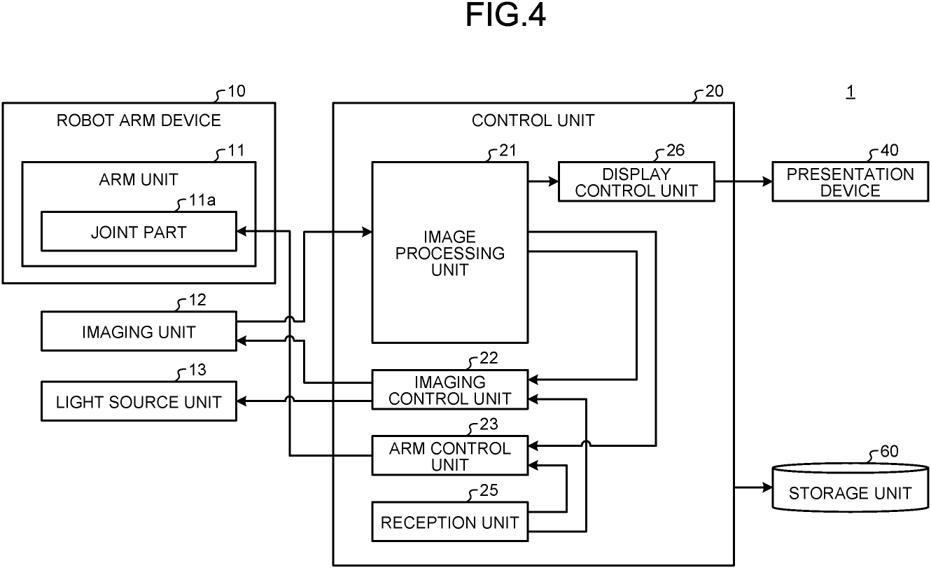

Moreover, a configuration of a medical observation system 1 according to the embodiment of the present disclosure that can be combined with the above-described endoscopic surgery system 5000 will be described with reference to FIG. 4. FIG. 4 is a block diagram illustrating an example of a configuration of the medical observation system 1 according to the embodiment of the present disclosure. As illustrated in FIG. 4, the medical observation system 1 mainly includes a robot arm device 10, an imaging unit 12, a light source unit 13, a control unit 20, a presentation device 40, and a storage unit 60. Hereinafter, each functional unit included in the medical observation system 1 will be described.

First, before describing the details of the configuration of the medical observation system 1, an outline of processing of the medical observation system 1 will be described. In the medical observation system 1, first, for example, the above-described endoscope 5001 (corresponding to the imaging unit 12 in FIG. 4) is inserted into the body of the patient through a medical puncture device called a trocar, and the operator 5067 performs the laparoscopic surgery while image-capturing an area of interest. At this time, by driving the robot arm device 10, the endoscope 5001 can freely change an imaging position.

### Robot Arm Device 10

The robot arm device 10 includes an arm unit 11 (articulated arm) that is a multilink structure including a plurality of joint parts and a plurality of links, and drives the arm unit within a movable range to control the position and attitude of a distal end unit provided at a distal end of the arm unit. The robot arm device 10 corresponds to the support arm device 5027 illustrated in FIG. 1.

The robot arm device 10 can include, for example, the CCU 5039 illustrated in FIG. 2, an electronic cutout control unit (not illustrated) that cuts out a predetermined region from an image obtained by imaging an imaging target received from the CCU 5039 and outputs the cutout region to a GUI generation unit to be described later, an attitude control unit (not illustrated) that controls a position and attitude of the arm unit 11, and a GUI generation unit (not illustrated) that generates image data obtained by performing various types of processing on the image cut out by the electronic cutout control unit.

In the robot arm device 10 according to the embodiment of the present disclosure, the electronic degree of freedom of changing the line of sight by cutting out the captured image (wide angle/cutout function) and the degree of freedom by the actuator of the arm unit 11 are all treated as the degrees of freedom of the robot. As a result, it is possible to realize motion control in which the electronic degree of freedom of changing the line of sight and the degree of freedom of the joint by the actuator are linked.

Specifically, the arm unit 11 is a multilink structure including the plurality of joint parts and the plurality of links, and its driving is controlled by control from an arm control unit 23 to be described later. The arm unit 11 corresponds to the arm unit 5031 illustrated in FIG. 1. In FIG. 4, a plurality of joint parts are represented as one joint part 111. Specifically, the joint part 111 rotatably connects the links in the arm unit 11, and drives the arm unit 11 by controlling rotational driving thereof under the control of the arm control unit 23. Furthermore, the arm part 11 may include a motion sensor (not illustrated) including an acceleration sensor, a gyro sensor, a geomagnetic sensor, and the like in order to obtain information on the position and attitude of the arm unit 11.

### Imaging Unit 12

The imaging unit (medical observation device) 12 is provided at the distal end of the arm unit (medical arm) 11, and captures images of various imaging targets. That is, the arm unit 11 supports the imaging unit 12. As described above, the imaging unit 12 may be, for example, the stereo endoscope 100, an oblique endoscope (not illustrated), a front direct endoscope (not illustrated), an endoscope with a simultaneous imaging function in other directions (not illustrated), or a microscope, and is not particularly limited.

Moreover, the imaging unit 12 captures, for example, a surgical field image including various medical instruments, organs, and the like in the abdominal cavity of the patient. Specifically, the imaging unit 12 is a camera or the like capable of capturing an imaging target in a form of a moving image or a still image. More specifically, the imaging unit 12 is a wide-angle camera including a wide-angle optical system. For example, while an angle of view of a normal endoscope is about 80°, an angle of view of the imaging unit 12 according to the present embodiment may be 140°. Note that the angle of view of the imaging unit 12 may be smaller than 140° or may be 140° or more as long as it exceeds 80°. The imaging unit 12 transmits an electric signal (pixel signal) corresponding to a captured image to the control unit 20. Furthermore, the arm unit 11 may support a medical instrument such as the forceps 5023.

Furthermore, in the embodiment of the present disclosure, in a case where an endoscope other than the stereo endoscope 100 is used as the imaging unit 12 instead of the stereo endoscope 100 capable of distance measurement, a depth sensor (distance measuring device) (not illustrated) may be provided separately from the imaging unit 12. In this case, the imaging unit 12 can be a monocular endoscope. Specifically, the depth sensor can be, for example, a sensor that performs distance measurement using a time of flight (ToF) method in which distance measurement is performed using a return time of reflection of pulsed light from a subject, or a structured light method in which distance measurement is performed by distortion of a pattern by emitting lattice-shaped pattern light. Alternatively, in the present embodiment, the imaging unit 12 itself may be provided with a depth sensor. In this case, the imaging unit 12 can perform distance measurement by the ToF method simultaneously with imaging. Specifically, the imaging unit 12 includes a plurality of light receiving elements (not illustrated), and can generate an image or calculate distance information on the basis of a pixel signal obtained from the light receiving elements.

### Light Source Unit 13

In the light source unit 13, the imaging unit 12 irradiates the imaging object with light. The light source unit 13 can be realized by, for example, a light emitting diode (LED) for a wide angle lens. For example, the light source unit 13 may be configured by combining a normal LED and a lens to diffuse light. Furthermore, the light source unit 13 may have a configuration in which light transmitted through an optical fiber (light guide) is diffused (widened) by a lens. Furthermore, the light source unit 13 may expand an irradiation range by irradiating the optical fiber itself with light in a plurality of directions.

### Control Unit 20

The control unit 20 is implemented by, for example, a central processing unit (CPU), a micro processing unit (MPU), or the like executing a program (for example, a program according to an embodiment of the present disclosure) stored in the storage unit 60 described later using a random access memory (RAM) or the like as a work area. Furthermore, the control unit 20 is a controller, and may be realized by, for example, an integrated circuit such as an application specific integrated circuit (ASIC) or a field programmable gate array (FPGA). Specifically, the control unit 20 mainly includes an image processing unit 21, an imaging control unit 22, an arm control unit 23, a reception unit 25, and a display control unit 26.

The image processing unit 21 executes various processing on the imaging object captured by the imaging unit 12. Specifically, the image processing unit 21 acquires an image of the imaging object captured by the imaging unit 12, and generates various images on the basis of the image captured by the imaging unit 12. Specifically, the image processing unit 21 can generate an image by cutting out and enlarging a display target area (cutout range) in the image captured by the imaging unit 12. In this case, the image processing unit 21 may change a position (cutout range) where the image is cut out according to, for example, the state of the image captured by the imaging unit 12.

The imaging control unit 22 controls the imaging unit 12. For example, the imaging control unit 22 controls the imaging unit 12 to image the surgical field. The imaging control unit 22 controls, for example, a magnification of the imaging unit 12. Furthermore, for example, the imaging control unit 22 may control the enlargement magnification of the imaging unit 12 on the basis of the input information from the operator 5067 received by the reception unit 25, or may control the enlargement magnification of the imaging unit 12 according to the state of the image captured by the imaging unit 12, the state of display, or the like. Furthermore, the imaging control unit 22 may control the focus (focal length) of the imaging unit 12 or may control the gain (sensitivity) of the imaging unit 12 (specifically, the image sensor of the imaging unit 12) according to the state of the image captured by the imaging unit 12 or the like.

Furthermore, the imaging control unit 22 controls the light source unit 13. For example, the imaging control unit 22 controls the brightness of the light source unit 13 when the imaging unit 12 images the surgical field. For example, the imaging control unit 22 controls the brightness of the light source unit 13 on the basis of input information from the operator 5067 received by the reception unit 25.

The arm control unit 23 integrally controls the robot arm device 10 and controls driving of the arm unit 11. Specifically, the arm control unit 23 controls the driving of the arm unit 11 by controlling the driving of the joint part 11a. More specifically, the arm control unit 23 controls the number of rotations of the motor by controlling an amount of current supplied to the motor in the actuator of the joint part 11a, and controls the rotation angle and the generated torque in the joint part 11a.

The arm control unit 23 can autonomously control the attitude (position, angle) of the arm unit 11 according to, for example, the state of the image captured by the imaging unit 12.

The reception unit 25 can receive input information input from the operator 5067 and various input information (sensing data) from other devices (for example, a depth sensor or the like) and output the input information to the imaging control unit 22 and the arm control unit 23.

The display control unit 26 causes the presentation device 40 to be described later to display various images. For example, the display control unit 26 causes the presentation device 40 to display the image acquired from the imaging unit 12.

### Presentation Device 40

The presentation device 40 displays various images. The presentation device 40 displays, for example, an image captured by the imaging unit 12. The presentation device 40 can be, for example, a display including a liquid crystal display (LCD), an organic electro-luminescence (EL) display, or the like.

### Storage Unit 60

The storage unit 60 stores various types of information. The storage unit 60 is realized by, for example, a semiconductor memory element such as a random access memory (RAM) or a flash memory, or a storage device such as a hard disk or an optical disk.

### 3. Background to Creating Embodiments of Present Disclosure

Incidentally, the arm unit (medical arm) 11 supporting the imaging unit 12 described above can be autonomously moved using, for example, three-dimensional information in the abdominal cavity of the patient or recognition information of various medical instruments located in the abdominal cavity. However, the movement of the arm unit 11 (specifically, the distal end of the arm unit 11) may interfere with a medical instrument, an organ, or the like. Here, the interference means that the visual field of the imaging unit 12 is blocked by a non-target object (organ, tissue), a medical instrument, or the like, or that the imaging unit 12 itself collides with an organ, a tissue, a medical instrument, or the like. Therefore, in order to avoid such interference, it is conceivable that instead of moving the distal end of the arm unit 11, the abdominal cavity is imaged in advance at a wide angle, and an image obtained by moving a range (cutout range) of an image cut out from the acquired wide-angle image is presented to the operator 5067. Since the image presented to the operator 5067 appears to move by moving the range from which the image is cut out in this manner, the operator 5067 recognizes as if the distal end of the arm unit 11 moves up and down, left and right, for example (the distal end of the arm unit 11 moves virtually). In addition, since the distal end of the arm unit 11 does not actually move, interference due to movement of the distal end of the arm unit 11 can be avoided.

However, in the endoscope used as the imaging unit 12 so far, since the angle of view is narrow (For example, the horizontal view angle is about 70°.), there is a limit to freely moving a range (cutout range) in which an image is cut out as described above. In other words, the range in which the distal end of the arm unit 11 can virtually move is limited. Therefore, the present inventors have conceived of using an endoscope (For example, a horizontal view angle is about 140°.) (Hereinafter, also referred to as a wide-angle endoscope.) having a wide angle of view in order to secure a wide movable region of the cutout range.

However, since the distortion of the image of the end part in the wide-angle image obtained by the wide-angle endoscope as described above increases, distortion correction is performed in consideration of presentation to the operator 5067 or being subjected to various types of image processing. However, as a result of intensive studies by the present inventors, it has been found that the image becomes dark and the range of the dark image becomes wide by performing the correction. That is, it is difficult to avoid the image at the end part in the wide-angle image from becoming dark. Therefore, in a case where the cutout range presented to the operator 5067 is an end part of the wide-angle image, a dark image that is difficult to visually recognize is presented to the operator 5067.

Accordingly, in such a case, it is also conceivable to improve the brightness of the image by increasing the intensity of light from the light source unit 13 or adjusting the light guide angle by the light guide 124. However, since there are limitations on the range in which the intensity of light is allowed to be increased and the range in which light can be uniformly guided by the light guide 124, there is also a limitation on increasing the brightness of the end part of the wide-angle image. That is, when the cutout range is the end part of the wide-angle image, there is a limit to improving the visibility of the image in the cutout range only by adjusting the irradiation light.

Furthermore, in a case where the cutout range presented to the operator 5067 is an end part of the wide-angle image, it is conceivable to increase the intensity of light or adjust the light guide angle by the light guide 124 according to the state of the image of the end part. However, in such a case, it becomes difficult to make the visibility of the wide-angle image suitable, and for example, when both the wide-angle image and the image of the cutout range are presented to the operator 5067, the visibility may deteriorate in the wide-angle image. Furthermore, in a case where the wide-angle image obtained in this manner is provided to various types of image processing, it becomes difficult to suitably perform the image processing. That is, it is difficult to achieve both improvement in visibility between the wide-angle image and the image in the cutout range.

Therefore, in view of such a situation, the present inventors have created an embodiment of the present disclosure in which the brightness (visibility) of the image in the cutout range can be made preferable even in a place where the desired cutout range is the end part of the wide-angle image. In the embodiment of the present disclosure created by the present inventors, in order to suitably adjust the brightness of the image in the cutout range, it is possible to suitably adjust the brightness of the image in the cutout range by adjusting the attitude (position, angle) of the arm unit 11 (specifically, the imaging unit 12 at the distal end of the arm unit 11) on the basis of light amount distributions of the wide-angle image (first image) and the image in the cutout range (second image). Moreover, in the embodiment of the present disclosure created by the present inventors, not only the attitude of the arm unit 11 but also the gain (sensitivity) of the image sensor of the imaging unit 12 may be adjusted, and further, the position of the cutout range may also be adjusted. By doing so, the visibility of the image in the cutout range can be made more suitable. In addition, according to the embodiment of the present disclosure, it is also possible to improve both the visibility in the wide-angle image and the image in the cutout range.

Moreover, in a case where the focus (focal length) is set near a center of the wide-angle image, the focus in the cutout range may not be set depending on the position of the cutout range. Specifically, in a case where a subject depth of the endoscope is shallow, the focus adjusted near the center of the wide-angle image may deviate at the end part of the wide-angle image. Therefore, in the embodiment of the present disclosure created by the present inventors, focus adjustment may be performed in addition to the above-described adjustment. By doing so, according to the embodiment of the present disclosure, it is also possible to improve both the visibility in the wide-angle image and the image in the cutout range. Hereinafter, details of the embodiments of the present disclosure created by the present inventors will be sequentially described.

### 4. First Embodiment

### 4.1 Detailed Configuration Example of Control System 2

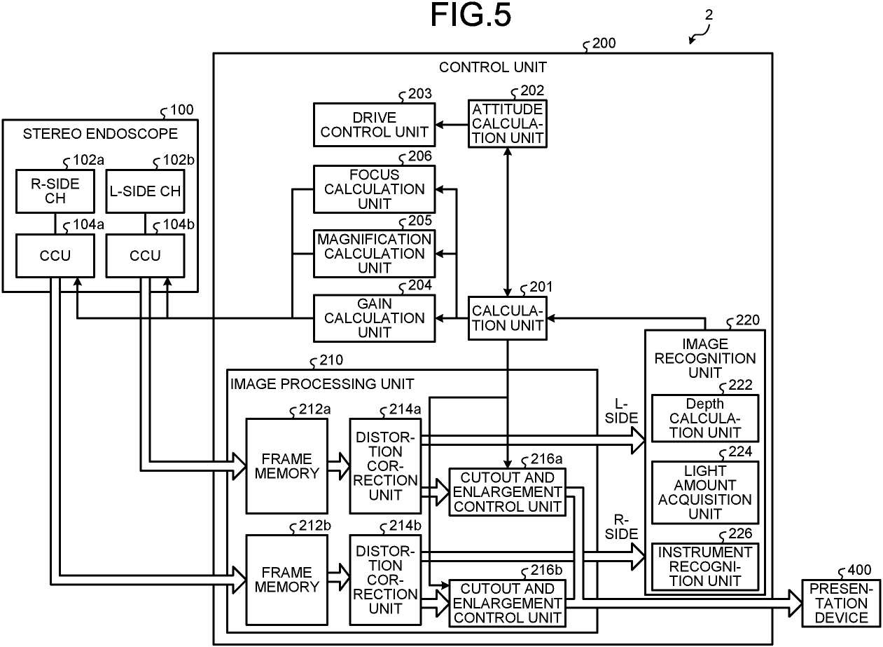

First, a detailed configuration example of a control system 2 according to a first embodiment of the present disclosure will be described with reference to FIG. 5. FIG. 5 is a block diagram illustrating an example of a configuration of the control system 2 according to the present embodiment. As illustrated in FIG. 5, the control system 2 according to the present embodiment can include a stereo endoscope 100, a control unit 200, and an arm unit 11 (not illustrated). Hereinafter, details of each device included in the control system 2 will be sequentially described.

### 4.2 Detailed Configuration Example of Stereo Endoscope 100

First, a detailed configuration example of the stereo endoscope 100 according to the present embodiment will be described with reference to FIG. 5. As described above, the stereo endoscope 100 can also perform distance measurement using the triangulation method, and includes a pair of image sensors (not illustrated) for acquiring right-eye (R-side) and left-eye (L-side) pixel signals corresponding to 3D display. The stereo endoscope 100 includes an R-side channel (CH) 102a that acquires a right-eye (R-side) pixel signal and an L-side channel 102b that acquires a left-eye (L-side) pixel signal. The pixel signal input to each of the channels 102a and 102b is output to each of camera control units (CCU) (sensor control unit, focus adjustment unit, magnification adjustment unit) 104a and 104b via a camera cable. Each CCU 104 can adjust the gain, focus, magnification, and the like of each image sensor of the stereo endoscope 100 according to the control from the control unit 200 described later. Note that the CCU 104 corresponds to the CCU 5039 illustrated in FIG. 1.

Furthermore, in the present embodiment, the stereo endoscope 100 is not limited to the form including the channels 102a and 102b that respectively acquire the pixel signals from the right-eye (R-side) and left-eye (L-side) image sensors as described above. For example, in the present embodiment, the stereo endoscope 100 may include a channel 102 that divides a pixel signal from one image sensor into two pixel signals for the right-eye (R-side) and the left-eye (L-side) and acquires the pixel signals.

### 4.3 Detailed Configuration Example of Control Unit 200

Next, a detailed configuration example of the control unit 200 according to the present embodiment will be described with reference to FIG. 5. Note that the control unit 200 may be included in the control unit 20 illustrated in FIG. 4, may be a device different from the control unit 20, or may be a device provided on a cloud and communicably connected to the robot arm device 10 and the control unit 20.

Specifically, as illustrated in FIG. 5, the control unit 200 mainly includes a calculation unit (range setting unit) 201, an attitude calculation unit (attitude recognition unit, attitude determination unit) 202, a drive control unit (arm control unit) 203, a gain calculation unit (gain determination unit) 204, a magnification calculation unit 205, a focus calculation unit 206, an image processing unit 210, and an image recognition unit 220. Hereinafter, details of each functional unit of the control unit 200 will be sequentially described.

### Calculation Unit 201

The calculation unit 201 can set a cutout range for cutting out an image (second image) having a narrower angle of view than the wide-angle image from the wide-angle image (first image) corrected by the image processing unit 210 acquired via the image recognition unit 220. For example, the image of the cutout range includes an image of an area (for example, a surgical site) in the body in which the operator 5067 is interested. For example, the calculation unit 201 can set the cutout range on the basis of information obtained by the input from the operator 5067, a distance (distance information) between the stereo endoscope 100 and an area (subject) of interest of the operator 5067, an attitude (position, angle) of the stereo endoscope 100 (arm unit 11), and information of the medical instrument or the like used by the operator 5067. Then, the calculation unit 201 outputs the set cutout range to the attitude calculation unit 202, the gain calculation unit 204, the magnification calculation unit 205, the focus calculation unit 206, and the image processing unit 210 described later.

### Attitude Calculation Unit 202

The attitude calculation unit 202 can recognize the attitude (position, angle) of the stereo endoscope 100 (arm unit 11). For example, the attitude calculation unit 202 can recognize the attitude of the stereo endoscope 100 on the basis of the wide-angle image (first image) corrected by the image processing unit 210 acquired via the image recognition unit 220. For example, the attitude calculation unit 202 may recognize the attitude of the stereo endoscope 100 on the basis of the wide-angle image using simultaneous localization and mapping (SLAM), for example. Alternatively, the attitude calculation unit 202 may recognize the attitude of the stereo endoscope 100 on the basis of distance information (for example, depth information) acquired via the image recognition unit 220 or sensing data from a motion sensor (inertial measurement device) provided in the arm unit 11. Alternatively, the attitude calculation unit 202 may recognize the attitude of the stereo endoscope 100 on the basis of joint angles and link lengths of the joint part 5033 and the link 5035 (a plurality of elements) included in the arm unit 11.

The attitude calculation unit 202 can determine the target attitude (position, angle) of the stereo endoscope 100 (arm unit 11) on the basis of a light amount distribution in the wide-angle image (first image) and the image in the cutout range (second image) obtained by the image recognition unit 220 and the distance information. Specifically, the attitude calculation unit 202 determines a target attitude of the stereo endoscope 100 so as to avoid overexposure (saturation) and darkening in the wide-angle image and the image of the cutout range. At this time, the attitude calculation unit 202 can determine the attitude of the stereo endoscope 100 corresponding to the image of the cutout range by specifying the position of the pixel in the image corresponding to the current attitude of the stereo endoscope 100 (position, angle). Then, the attitude calculation unit 202 outputs the determined target attitude to the drive control unit 203.

### Drive Control Unit 203

The drive control unit 203 can control the attitude (position, angle) of the stereo endoscope 100 (arm unit 11) on the basis of the target attitude from the attitude calculation unit 202.

### Gain Calculation Unit 204

The gain calculation unit 204 can determine a target gain (target sensitivity) of the image sensor of the stereo endoscope 100 (arm unit 11) on the basis of the light amount distributions in the wide-angle image (first image) and the image in the cutout range (second image) obtained by the image recognition unit 220 and the distance information. Specifically, the gain calculation unit 204 determines the target gain of the image sensor of the stereo endoscope 100 so as to avoid overexposure (saturation) and darkening in the wide-angle image and the image in the cutout range. Then, the gain calculation unit 204 outputs the determined target gain to the CCU 104.

### Magnification Calculation Unit 205

The magnification calculation unit 205 can calculate a suitable magnification of the image of the cutout range to be presented to the operator 5067. The magnification calculation unit 205 outputs the calculated enlargement magnification to the CCU 104 and the image processing unit 210, and converts the image in the cutout range into the image with the enlargement magnification obtained by the calculation, so that the visibility of the image in the cutout range can be improved. For example, in a case where the attitude of the stereo endoscope 100 (arm unit 11) is adjusted to the target attitude, if the enlargement magnification of the image in the cutout range is maintained as it is, a subject in the image in the cutout range becomes small, and the visibility of the subject may deteriorate. Therefore, the magnification calculation unit 205 can improve the visibility of the image in the cutout range by calculating a suitable magnification on the basis of the size of the subject or the like and converting the image in the cutout range into the image with the magnification obtained by the calculation. Note that, in a second embodiment of the present disclosure described later, details of an operation of the magnification calculation unit 205 will be described.

### Focus Calculation Unit 206

The focus calculation unit 206 calculates a suitable focus (focal length) for the wide-angle image and the image in the cutout range, and controls the stereo endoscope 100 so as to obtain the focus obtained by the calculation, so that it is possible to achieve both a suitable focus for the wide-angle image and the image in the cutout range. Specifically, when the focus is set near the center of the wide-angle image, the focus may not be set in the cutout range depending on the position of the cutout range. Therefore, the focus calculation unit 206 calculates a suitable focus for the wide-angle image and the image in the cutout range, and controls the stereo endoscope 100 to the focus obtained by the calculation, thereby achieving both the focus of the wide-angle image and the focus of the image in the cutout range. Note that, in the second embodiment of the present disclosure described later, details of an operation of the focus calculation unit 206 will be described.

### Image Processing Unit 210

As illustrated in FIG. 5, the image processing unit 210 includes frame memories 212a and 212b, distortion correction units 214a and 214b, and cutout and enlargement control units 216a and 216b. Specifically, the frame memories 212a and 212b can store right-eye (R-side) and left-eye (L-side) image signals from the CCUs 104a and 104b, respectively, and can output the stored image signals to the distortion correction units 214a and 214b, respectively.

The distortion correction units 214a and 214b can correct lens distortion in the right-eye (R-side) and left-eye (L-side) image signals from the frame memories 212a and 212b, respectively. As described above, the lens distortion is large at the end part of the wide-angle image, and when the distortion is large, the accuracy of the subsequent processing (depth calculation, image recognition, setting of a cutout range, etc.) decreases. Therefore, in the present embodiment, the distortion is corrected. Then, the distortion correction units 214a and 214b output the corrected image signal to the cutout and enlargement control units 216a and 216b and the image recognition unit 220 described later.

The cutout and enlargement control units 216a and 216b acquire an image in the cutout range on the basis of the corrected image signal and the cutout range set by the calculation unit 201, and output the image to the presentation device 40.

### Image Recognition Unit 220

As illustrated in FIG. 5, the image recognition unit 220 includes a depth calculation unit (distance acquisition unit) 222, a light amount acquisition unit 224, and an instrument recognition unit 226. Specifically, the depth calculation unit 222 acquires distance information between the stereo endoscope (medical observation device) 100 and the subject. For example, in the stereo endoscope 100, as described above, since the pixel signals for the right-eye and the left-eye are acquired, not only an image corresponding to 3D display can be obtained, but also distance measurement can be performed using the triangulation method. Therefore, in the present embodiment, the depth calculation unit 222 can acquire the distance information between the stereo endoscope 100 and the subject on the basis of the wide-angle image for the right-eye and the wide-angle image (image signal) for the left-eye by the stereo endoscope 100 from the image processing unit 210. Furthermore, in the present embodiment, the depth calculation unit 222 may acquire distance information between the stereo endoscope 100 and the subject on the basis of sensing data from a depth sensor (distance measuring device) such as a ToF sensor or a structured light provided at the distal end of the arm unit 11 or the like. Then, the depth calculation unit 222 outputs the acquired distance information to the calculation unit 201.

Based on the wide-angle image (first image) from the image processing unit 210, the light amount acquisition unit 224 acquires a light amount distribution in the wide-angle image and a light amount distribution of the image (second image) in the cutout range, and outputs the light amount distributions to the calculation unit 201. Specifically, the light amount acquisition unit 224 acquires the light amount distribution of the image in the cutout range from the light amount distribution in the wide-angle image.

The instrument recognition unit 226 can recognize the medical instrument inserted into the abdominal cavity by extracting a contour or the like of the subject from the wide-angle image from the image processing unit 210 and comparing the extracted contour with data stored in advance in a storage unit (not illustrated). Then, the instrument recognition unit 226 outputs a recognition result to the calculation unit 201.

Note that, in the present embodiment, each functional unit of the control unit 200 is not limited to the functional unit illustrated in FIG. 5.

### 4.4 Control Method

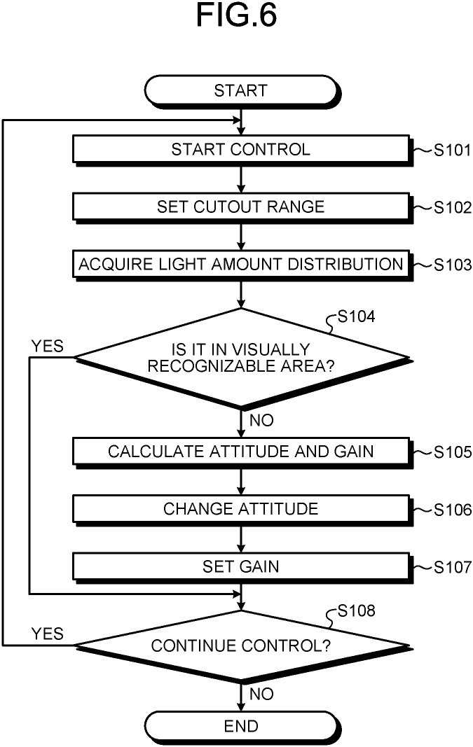

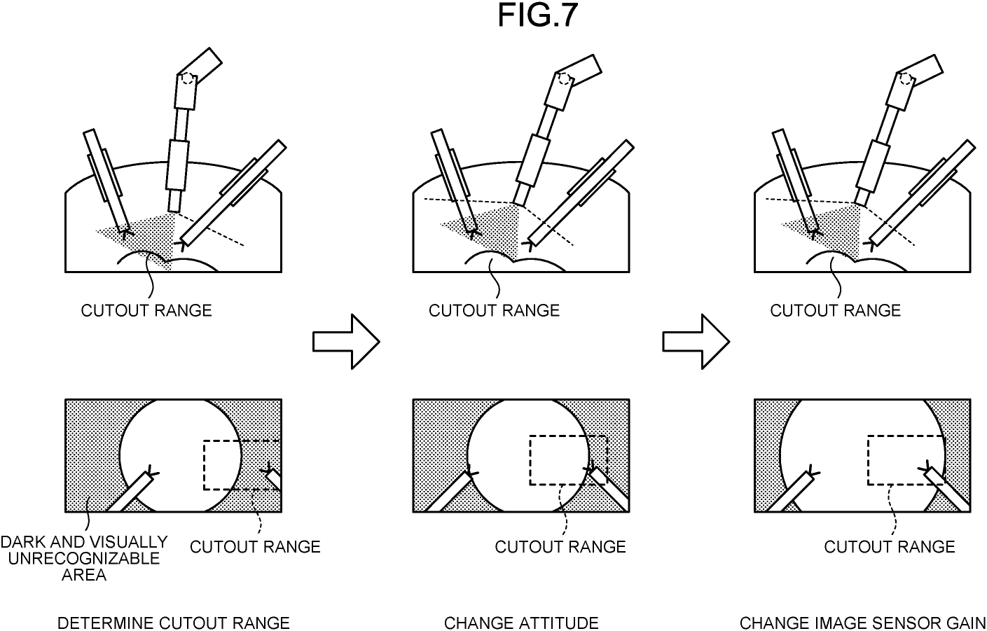

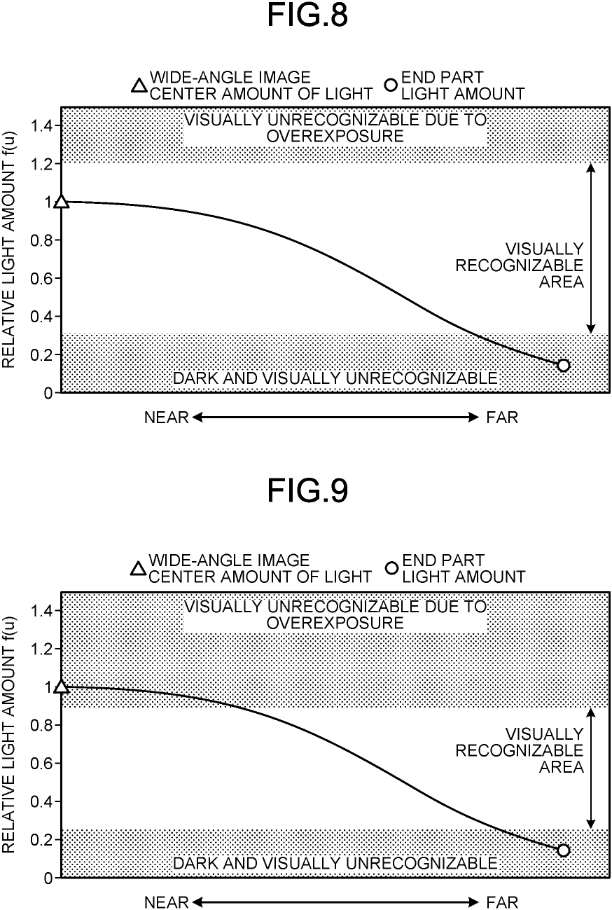

Next, a control method according to the present embodiment will be described with reference to FIGS. 6 to 11. FIG. 6 is a flowchart of the control method according to the present embodiment, and FIG. 7 is an explanatory diagram for describing the present embodiment. Furthermore, FIGS. 8 to 11 are graphs for explaining the control method in the present embodiment, and specifically, are graphs illustrating a relationship between a distance from the center of the wide-angle image and the light amount.

As illustrated in FIG. 6, the control method according to the present embodiment can mainly include Steps from Step S101 to Step S108. Details of these steps according to the present embodiment will be described below.

First, the control system 2 starts the control according to the present embodiment (Step S101). Next, the control system 2 recognizes, for example, a medical instrument inserted into the abdominal cavity, and further acquires distance information between the stereo endoscope (medical observation device) 100 and the medical instrument. Then, the control system 2 changes the setting of the cutout range and the position of the stereo endoscope 100 (arm unit 11) based on the recognized information on the medical instrument and the distance information (Step S102). At this time, for example, it is assumed that a wide-angle image as illustrated on a lower left side of FIG. 7 is obtained, and a cutout range located at an end part of the wide-angle image as illustrated on the lower left side of FIG. 7 is set.

Next, based on the wide-angle image (first image) from the image processing unit 210, the control system 2 acquires a light amount distribution in the wide-angle image and a light amount distribution in the image (second image) in the cutout range (Step S103).

For example, FIG. 8 illustrates amounts of light at the central part and the end part of the wide-angle image. Specifically, a horizontal axis indicates a distance from the center of the wide-angle image, and a vertical axis indicates a relative amount of light in a case where an amount of light at the central part (indicated by a triangular mark in FIG. 8) is 1 in a case where a distance (Hereinafter, referred to as WD) between the stereo endoscope 100 and the subject (for example, a medical instrument) is 50 mm. Furthermore, an area on an upper side of the graph of FIG. 8 indicates an area in which the image is overexposed due to too high light amount and cannot be visually recognized, and an area on a lower side of the graph of FIG. 8 indicates an area in which the image is dark due to too low light amount and cannot be visually recognized. An area sandwiched between these two areas is a visually recognizable area.

Specifically, in the state illustrated on a left side of FIG. 7, the cutout range is located, for example, at the end part of the wide-angle image. Therefore, in FIG. 8, a light amount of the image in the cutout range indicated by the circle is too low, so that the image is dark and enters an area that cannot be visually recognized.

Therefore, the control system 2 determines whether the light amount of the image in the cutout range is within the visually recognizable area illustrated in FIG. 8 (Step S104). The control system 2 proceeds to Step S108 when the light amount of the image in the cutout range is within the visually recognizable area (Step S104: Yes), and proceeds to Step S105 when the light amount of the image in the cutout range is not within the visually recognizable area (Step S104: No).

Next, the control system 2 calculates and determines the target attitude (position and angle) of the stereo endoscope 100 (arm unit 11) and further calculates and determines the target gain (target sensitivity) of the image sensor of the stereo endoscope 100 so that the amounts of light of both the central part of the wide-angle image and the image in the cutout range enter the visually recognizable area (Step S105).

Note that, in the present embodiment, the target attitude of the stereo endoscope 100 (arm unit 11) is adjusted in addition to the adjustment of the target gain for the following reasons. For example, in a case where the gain of the image sensor is adjusted to 3 dB (1.33 times), the visually recognizable area changes from the state of FIG. 8 to the state of FIG. 9. As illustrated in FIG. 9, by increasing the gain, the light amount is too low, so that the image is dark, and a threshold of the area that cannot be visually recognized (the boundary with the visually recognizable area) decreases, but the light amount is too high, so that the image is overexposed, and the threshold of the area that cannot be visually recognized (the boundary with the visible area) also decreases. Therefore, as is clear from FIG. 9, the light amount (indicated by the triangle mark) at the central part of the wide-angle image and the light amount (indicated by the circle mark) at the end part of the wide-angle image enter the visually unrecognizable areas. Therefore, in the present embodiment, not only the gain of the image sensor but also the attitude (position, angle) of the stereo endoscope 100 (arm unit 11) is adjusted so that the light amounts of both the central part of the wide-angle image and the image in the cutout range enter the visually recognizable area.

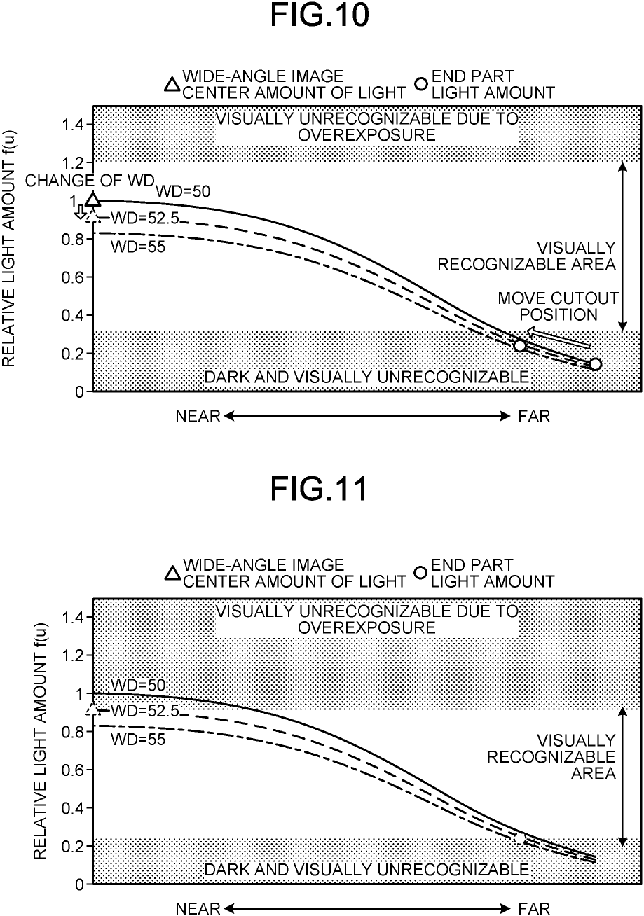

Next, the control system 2 changes the attitude (position, angle) of the stereo endoscope 100 (arm unit 11) according to the target attitude determined in Step S105 (Step S106). By changing the attitude of the stereo endoscope 100, a wide-angle image and a cutout range as illustrated in the center of FIG. 7 are changed. More specifically, for example, as illustrated in FIG. 10 (Here, a graph in a case where the gain is set to 0 dB is illustrated.), by changing an insertion amount of the stereo endoscope 100 into the body and increasing a distance WD between the stereo endoscope 100 and the subject (for example, a medical instrument) (changing from WD = 50 to WD = 52.5), the light amount (indicated by the triangular mark) at the central part of the wide-angle image decreases. In this way, the amount of light at the central part of the wide-angle image is suppressed from entering an area where the image cannot be visually recognized due to overexposure of the image due to the too high amount of light. On the other hand, for the image in the cutout range, the attitude of the stereo endoscope 100 is changed, and the cutout range is brought close to the central part of the wide-angle image, so that the amount of light (indicated by a circle) of the image in the cutout range is suppressed from entering an area where the image is dark and cannot be visually recognized because the amount of light is too low. However, at this time point, since the gain of the image sensor is not adjusted, all the light amounts of the images in the cutout range are not in the visually recognizable area (see a central diagram in FIG. 7).

Next, the control system 2 sets a gain (sensitivity) of the image sensor according to the target gain determined in Step S105 (Step S107). By changing the gain of the image sensor, the image is changed to a wide-angle image and a cutout range as illustrated on a right side of FIG. 7. More specifically, for example, as illustrated in FIG. 11, in a case where the gain of the image sensor is changed to 3 dB (1.33 times), the visually recognizable area changes from the state of FIG. 10 to the state of FIG. 11. Therefore, the light amount (indicated by the circle) of the image in the cutout range that has not entered the visible area enters the visually recognizable area in the entire range by increasing the gain. That is, in the present embodiment, by adjusting the attitude (position, angle) of the stereo endoscope 100 (arm unit 11) and the gain (sensitivity) of the image sensor, it is possible to allow the light amounts of both the central part of the wide-angle image and the image in the cutout range to enter the visually recognizable area. As a result, according to the present embodiment, it is possible to improve the visibility of both the central part of the wide-angle image and the image in the cutout range.

Note that the present embodiment is not limited to adjusting both the attitude (position, angle) of the stereo endoscope 100 (arm unit 11) and the gain (sensitivity) of the image sensor. In the present embodiment, the adjustment of the gain of the image sensor may be omitted as long as the light amounts of both the central part of the wide-angle image and the image in the cutout range can be made to enter the visually recognizable area only by the adjustment of the attitude of the stereo endoscope 100.

Then, the control system 2 determines whether to continue the control according to the present embodiment (Step S108). The control system 2 returns to Step S101 when determining to continue (Step S108: Yes), and terminates the control according to the present embodiment when determining not to continue (Step S108: No).

As described above, in the present embodiment, the attitude of the stereo endoscope 100 (arm unit 11) is adjusted on the basis of the light amount distributions of the wide-angle image (first image) and the image in the cutout range (second image), whereby the brightness of the image in the cutout range can be suitably set. Moreover, in the present embodiment, not only the attitude of the stereo endoscope 100 (arm unit 11) but also the gain (sensitivity) of the image sensor of the imaging unit 12 may be adjusted and the cutout range may be moved. In this way, according to the present embodiment, the brightness of the wide-angle image and the image in the cutout range is suitable, and it is possible to improve both the visibility of the wide-angle image and the image in the cutout range.

### 5. Second Embodiment

In the first embodiment described above, the focus (focal length) is not automatically adjusted, but in the embodiment of the present disclosure, the focus may also be automatically adjusted. For example, as described above, in a case where the subject depth of the stereo endoscope 100 is shallow, when focusing is performed near the center of the wide-angle image, there is a case where focusing is not performed in the cutout range located at the end part of the wide-angle image. Therefore, in the second embodiment of the present disclosure, focus adjustment is performed in addition to the adjustment in the first embodiment. By doing so, according to the present embodiment, it is possible to make the visibility of the image in the cutout range more preferable.

Specifically, for example, in the stereo endoscope 100 having an angle of view of 140° (half angle θ = 70°), when an object on a plane is to be imaged, a distance W_{edge} from the center to the end part of the obtained image can be expressed by the following Formula (1).

$${W}_{e d g e} = \frac{W D}{cos 70 °} \qquad \text{(1)}$$

For example, in a case where the distance WD between the stereo endoscope 100 and the subject (for example, a medical instrument) is 50 mm, the distance W_{edge} from the center to the end part of the image is 146.2 mm with reference to Formula (1). Therefore, assuming that the subject depth of the stereo endoscope 100 is 50to 85 mm, in a case where the distance WD is 50 mm, even if focus is achieved near the center of the image, there is a possibility that focus is not achieved since the maximum value of the subject depth exceeds 85 mm at the end part of the image (distance W_{edge} = 146.2 mm). Therefore, in the present embodiment, focus adjustment is performed in addition to the adjustment in the first embodiment. Hereinafter, details of the present embodiment will be sequentially described.

### 5.1 Detailed Configuration Example of Control System 2, Stereo Endoscope 100, and Control Unit 200

Note that a control system 2, a stereo endoscope 100, and a control unit 200 according to the present embodiment are common to the control system 2, the stereo endoscope 100, and the control unit 200 according to the first embodiment, and thus, description of detailed configurations of the control system 2, the stereo endoscope 100, and the control unit 200 according to the present embodiment will be omitted here.

### 5.2 Control Method

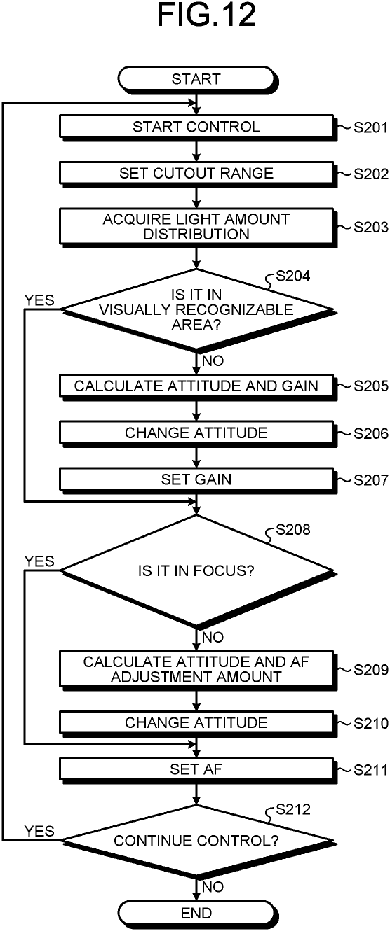

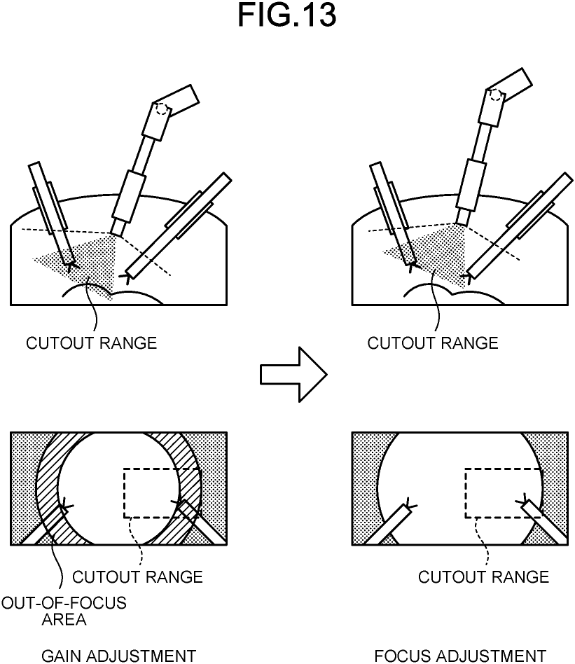

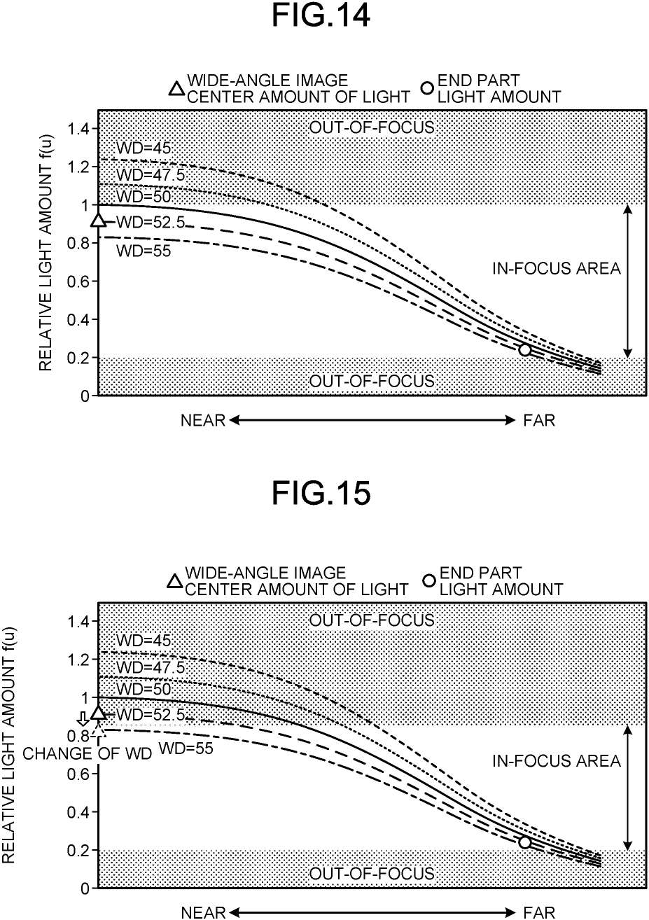

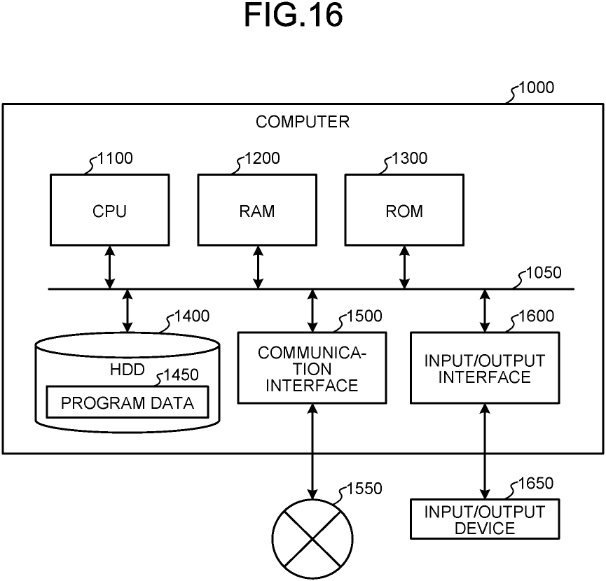

Next, a control method according to the present embodiment will be described with reference to FIGS. 12 to 15. FIG. 12 is a flowchart of the control method according to the present embodiment, and FIG. 13 is an explanatory diagram for describing the present embodiment. Furthermore, FIGS. 14 and 15 are graphs for describing the control method in the present embodiment, and specifically, are graphs illustrating a relationship between a distance from the center of the wide-angle image and a light amount.

Specifically, as illustrated in FIG. 12, the control method according to the present embodiment can mainly include steps from Step S201 to Step S212. Details of these steps according to the present embodiment will be described below.

First, since Step S201 to S207 illustrated in FIG. 12 are common to Step S101 to S107 of the control method according to the first embodiment illustrated in FIG. 6, the description of Step S201 to S207 is omitted here. Furthermore, at the time of Step S207, for example, it is assumed that a wide-angle image as illustrated on a lower left side of FIG. 13 is obtained.

Next, the control system 2 determines whether the central part of the wide-angle image and the cutout range are at a position where the focus is achieved (Step S208). The control system 2 proceeds to Step S211 when the central part of the wide-angle image and the cutout range are at a focused position (Step S208: Yes), and proceeds to Step S209 when the central part of the wide-angle image and the cutout range are not at the focused position (Step S208: No).

For example, in a case where the maximum value of the subject depth of the stereo endoscope 100 is 85 mm, when a focused position is expressed by a distance from the center of the wide-angle image (the distance is expressed by the number of pixels), the following Formula (2) is obtained.

$${P}_{f o c u s} = \frac{\sqrt{{85}^{2} - W {D}^{2}}}{W D ∗ t a n 70 °} ∗ 1920 \qquad \text{(2)}$$

For example, FIG. 14 illustrates the amounts of light at the central part and the end part of the wide-angle image. Specifically, a horizontal axis indicates a distance from the center of the wide-angle image, and a vertical axis indicates a relative amount of light in a case where an amount of light at the central part (indicated by a triangular mark) is 1. Furthermore, an upper area of the graph of FIG. 14 indicates an out-of-focus area, and a lower area of the graph of FIG. 14 also indicates an out-of-focus area. An area sandwiched between these two areas is an area in which focus is achieved.

Specifically, in the state illustrated on a left side of FIG. 13, the cutout range is located at the end part of the wide-angle image, and thus, in FIG. 14, the cutout range indicated by a circle has entered the out-of-focus area.

Therefore, the control system 2 calculates and determines a target attitude (position, angle) of the stereo endoscope 100 (arm unit 11) and further calculates and determines an adjustment amount of the focus of the image sensor of the stereo endoscope 100 so as to be a position where the focus is achieved in both the central part of the wide-angle image and the cutout range (Step S209).

Next, the control system 2 changes the attitude (position, angle) of the stereo endoscope 100 (arm unit 11) according to the target attitude determined in Step S209 (Step S210). Specifically, the attitude of the stereo endoscope 100 is changed to a wide-angle image and a cutout range as illustrated on a right side of FIG. 13. More specifically, for example, as illustrated in FIG. 15, by changing an insertion amount of the stereo endoscope 100 into the body and increasing a distance WD between the stereo endoscope 100 and the subject (for example, a medical instrument) (changing from WD = 52.5 to WD = 55), both the central part (indicated by a triangular mark) and the cutout range (indicated by a circle mark) of the wide-angle image become the focused position.

Next, the control system 2 sets the focus of the image sensor according to the focus adjustment amount determined in Step S209 (Step S211).

Note that the present embodiment is not limited to adjusting both the attitude (position, angle) of the stereo endoscope 100 (arm unit 11) and the focus of the image sensor. In the present embodiment, the adjustment of the focus of the image sensor may be omitted as long as it is possible to bring both the central part of the wide-angle image and the cutout range into a focused position only by the adjustment of the attitude of the stereo endoscope 100.

Moreover, since Step S212 illustrated in FIG. 12 is common to Step S108 of the control method according to the first embodiment illustrated in FIG. 6, the description of Step S212 is omitted here.

As described above, in the present embodiment, the focus adjustment is performed in addition to the adjustment in the first embodiment. With this configuration, according to the present embodiment, it is possible to further improve the visibility between the optical image and the image in the cutout range.

In the present embodiment, in a case where the attitude of the stereo endoscope 100 (arm unit 11) is adjusted to the target attitude, if the magnification of the image of the cutout range is maintained, a subject in the image of the cutout range becomes small, and the visibility of the subject may deteriorate. Therefore, in the present embodiment, the magnification calculation unit 205 can improve the visibility of the image in the cutout range by calculating a suitable enlargement magnification of the image in the surgical cutout range and converting the image in the cutout range into the image of the enlargement magnification obtained by the calculation.

More specifically, the magnification calculation unit 205 can calculate an enlargement magnification S of the image in the cutout range according to the following Formula (3) including a distance WD between the stereo endoscope 100 and the subject (for example, a medical instrument).

$$\begin{matrix}\text{S} = {S}_{d e f a u l t} \frac{W {D}_{c h a n g e}}{W {D}_{d e f a u l t}} \\ {S}_{d e f a u l t} : \text{Magnification in cutout range before attitude change} \\ W {D}_{d e f a u l t} : \text{WD before attitude change} \\ W {D}_{c h a n g e} : \text{WD after attitude change}\end{matrix} \qquad \text{(3)}$$

Then, the magnification calculation unit 205 can improve the visibility of the image in the cutout range by converting the image in the cutout range into the image with the enlargement magnification obtained by the calculation on the basis of the enlargement magnification S obtained by the calculation according to the above Formula (3).

### 6. Summary

As described above, in the embodiment of the present disclosure, the attitude of the stereo endoscope 100 (arm unit 11) is adjusted on the basis of the light amount distributions of the wide-angle image (first image) and the image in the cutout range (second image), whereby the brightness of the image in the cutout range can be suitably set. Therefore, according to the embodiment of the present disclosure, it is possible to further improve the visibility of the image in the cutout range.

### 7. Hardware Configuration

The information processing apparatus such as the control unit 200 according to each embodiment described above is realized by a computer 1000 having a configuration as illustrated in FIG. 16, for example. Hereinafter, the control unit 200 according to the embodiment of the present disclosure will be described as an example. FIG. 16 is a hardware configuration diagram illustrating an example of the computer 1000 that implements the functions of the control unit 200. The computer 1000 includes a CPU 1100, a RAM 1200, a read only memory (ROM) 1300, a hard disk drive (HDD) 1400, a communication interface 1500, and an input/output interface 1600. Each unit of the computer 1000 is connected by a bus 1050.

The CPU 1100 operates on the basis of a program stored in the ROM 1300 or the HDD 1400, and controls each unit. For example, the CPU 1100 develops a program stored in the ROM 1300 or the HDD 1400 in the RAM 1200, and executes processing corresponding to various programs.

The ROM 1300 stores a boot program such as a basic input output system (BIOS) executed by the CPU 1100 when the computer 1000 is activated, a program depending on hardware of the computer 1000, and the like.

The HDD 1400 is a computer-readable recording medium that non-transiently records a program executed by the CPU 1100, data used by the program, and the like. Specifically, the HDD 1400 is a recording medium that records a control program according to the present disclosure as an example of program data 1450.

The communication interface 1500 is an interface for the computer 1000 to connect to an external network 1550 (for example, the Internet). For example, the CPU 1100 receives data from other device or transmits data generated by the CPU 1100 to other device via the communication interface 1500.

The input/output interface 1600 is an interface for connecting the input/output device 1650 and the computer 1000. For example, the CPU 1100 receives data from an input device such as a keyboard and a mouse via the input/output interface 1600. Furthermore, the CPU 1100 transmits data to an output device such as a display, a speaker, or a printer via the input/output interface 1600. Furthermore, the input/output interface 1600 may function as a media interface that reads a program or the like recorded on a computer-readable predetermined recording medium (medium). The medium is, for example, an optical recording medium such as a digital versatile disc (DVD) or a phase change rewritable disk (PD), a magneto-optical recording medium such as a magneto-optical disk (MO), a tape medium, a magnetic recording medium, a semiconductor memory, or the like.

For example, in a case where the computer 1000 functions as the control unit 200 according to the embodiment of the present disclosure, the CPU 1100 of the computer 1000 executes the image processing program loaded on the RAM 1200 to implement the functions of the calculation unit 201 and the like. Furthermore, the HDD 1400 may store a control program according to the present disclosure and data in the storage unit 60. Note that the CPU 1100 reads the program data 1450 from the HDD 1400 and executes the program data. However, as another example, an information processing program may be acquired from another device via the external network 1550.

Furthermore, the control unit 200 according to the present embodiment may be applied to a system including a plurality of devices on the premise of connection to a network (or communication between devices), such as cloud computing. That is, the control unit 200 according to the present embodiment described above can be realized as the control system according to the present embodiment by, for example, a plurality of devices.

An example of the hardware configuration of the control unit 200 has been described above. Each of the above-described components may be configured using a general-purpose member, or may be configured by hardware specialized for the function of each component. Such a configuration can be appropriately changed according to the technical level at the time of implementation.

### 8. Supplement

Note that the embodiment of the present disclosure described above can include, for example, a control method executed by the control device or the control system as described above, a program for causing the control system or the control device to function, and a non-transitory tangible medium in which the program is recorded. Furthermore, the program may be distributed via a communication line (including wireless communication) such as the Internet.

Furthermore, each step in the control method of the embodiment of the present disclosure described above may not necessarily be processed in the described order. For example, each step may be processed in an appropriately changed order. Furthermore, each step may be partially processed in parallel or individually instead of being processed in time series. Moreover, the processing of each step does not necessarily have to be performed according to the described method, and may be performed by another method by another functional unit, for example.

Among the processes described in the above embodiments, all or a part of the processing described as being automatically performed can be manually performed, or all or a part of the processing described as being manually performed can be automatically performed by a known method. In addition, the processing procedure, specific name, and information including various data and parameters illustrated in the document and the drawings can be arbitrarily changed unless otherwise specified. For example, the various types of information illustrated in each figure are not limited to the illustrated information.

Furthermore, each component of each device illustrated in the drawings is functionally conceptual, and is not necessarily physically configured as illustrated in the drawings. That is, a specific form of distribution and integration of each device is not limited to the illustrated form, and all or a part thereof can be functionally or physically distributed and integrated in an arbitrary unit according to various loads, usage conditions, and the like.

Although the preferred embodiments of the present disclosure have been described in detail with reference to the accompanying drawings, the technical scope of the present disclosure is not limited to such examples. It is obvious that a person having ordinary knowledge in the technical field of the present disclosure can conceive various changes or modifications within the scope of the technical idea described in the claims, and it is naturally understood that these also belong to the technical scope of the present disclosure.

Furthermore, the advantageous effects described in the present specification are merely illustrative or exemplary, and are not restrictive. That is, the technique according to the present disclosure can exhibit other advantageous effects obvious to those skilled in the art from the description of the present specification together with or instead of the above advantageous effects.

Note that the present technique can also have the following configurations.

A medical arm control system comprising:
a range setting unit that sets a cutout range in which a second image is cut out from a first image by a medical observation device supported by an arm unit;
a light amount acquisition unit that acquires a light amount distribution of the first image and a light amount distribution of the second image;
a distance acquisition unit that acquires distance information indicating a distance between a subject of the medical observation device and the medical observation device; and
an arm control unit that controls the arm unit based on the light amount distributions of the first and second images and the distance information.

The medical arm control system according to (1), wherein the light amount acquisition unit acquires the light amount distribution of the second image from the light amount distribution of the first image.

The medical arm control system according to (1) or (2), further comprising

a sensor control unit that controls a gain of an image sensor of the medical observation device based on the light amount distributions of the first and second images and the distance information.

The medical arm control system according to any one of (1) to (3), wherein an angle of view of the second image is narrower than an angle of view of the first image.

The medical arm control system according to any one of (1) to (4), wherein the medical observation device is a wide-angle endoscope.

The medical arm control system according to any one of (1) to (5), wherein
the medical observation device is a stereo endoscope, and
the distance acquisition unit acquires the distance information based on two of the first images by the stereo endoscope.

The medical arm control system according to any one of (1) to (5), further comprising

a distance measuring device.

The medical arm control system according to (7), wherein the distance measuring device performs distance measurement using a ToF method or a structured light method.

The medical arm control system according to any one of (1) to (8), further comprising a correction unit that corrects distortion of the first image.

The medical arm control system according to any one of (1) to (9), further comprising a focus adjustment unit that automatically adjusts a focus of the medical observation device.

The medical arm control system according to any one of (1) to (10), further comprising a magnification adjustment unit that automatically adjusts a magnification of the second image.

The medical arm control system according to any one of (1) to (11), further comprising

an attitude recognition unit that recognizes an attitude of the arm unit.

The medical arm control system according to (12), wherein

the attitude recognition unit recognizes an attitude of the arm unit based on the first image.

The medical arm control system according to (12), wherein

the attitude recognition unit recognizes the attitude of the arm unit based on sensing data from an inertial measurement device provided in the arm unit or lengths and angles of a plurality of elements included in the arm unit.

The medical arm control system according to any one of (12) to (14), further comprising an instrument recognition unit that recognizes a medical instrument based on the first image.

The medical arm control system according to (15), wherein the range setting unit sets the cutout range for cutting out the second image based on at least one information of the distance information, the attitude of the arm unit, or the medical instrument.

The medical arm control system according to any one of (1) to (16), further comprising the arm unit.

A medical arm control method, by a medical arm control device, comprising:
setting a cutout range in which a second image is cut out from a first image by a medical observation device supported by an arm unit;
acquiring a light amount distribution of the first image and a light amount distribution of the second image;
acquiring distance information indicating a distance between a subject of the medical observation device and the medical observation device; and
controlling the arm unit based on the light amount distributions of the first and second images and the distance information.

A program causing a computer to function as:
a range setting unit that sets a cutout range in which a second image is cut out from a first image by a medical observation device supported by an arm unit;
a light amount acquisition unit that acquires a light amount distribution of the first image and a light amount distribution of the second image;
a distance acquisition unit that acquires distance information indicating a distance between a subject of the medical observation device and the medical observation device; and
an arm control unit that controls the arm unit based on the light amount distributions of the first and second images and the distance information.

### REFERENCE SIGNS LIST

1 MEDICAL OBSERVATION SYSTEM
2 CONTROL SYSTEM
10 ROBOT ARM DEVICE
11 ARM UNIT
11a JOINT PART
12 IMAGING UNIT
13 LIGHT SOURCE UNIT
20, 200 CONTROL UNIT
21, 210 IMAGE PROCESSING UNIT
22 IMAGING CONTROL UNIT
23 ARM CONTROL UNIT
25 RECEPTION UNIT
26 DISPLAY CONTROL UNIT
40 PRESENTATION DEVICE
60 STORAGE UNIT
100 STEREO ENDOSCOPE
102a, 102b CHANNEL
104a, 104b CCU
122a, 122b RELAY LENS
124 LIGHT GUIDE
201 CALCULATION UNIT
202 ATTITUDE CALCULATION UNIT
203 DRIVE CONTROL UNIT
204 GAIN CALCULATION UNIT
205 MAGNIFICATION CALCULATION UNIT
206 FOCUS CALCULATION UNIT
212a, 212b FRAME MEMORY
214a, 214b DISTORTION CORRECTION UNIT
216a, 216b CUTOUT AND ENLARGEMENT CONTROL UNIT
220 IMAGE RECOGNITION UNIT
222 DEPTH CALCULATION UNIT
224 LIGHT AMOUNT ACQUISITION UNIT
226 INSTRUMENT RECOGNITION UNIT

1. A medical arm control system comprising:
a range setting unit that sets a cutout range in which a second image is cut out from a first image by a medical observation device supported by an arm unit;
a light amount acquisition unit that acquires a light amount distribution of the first image and a light amount distribution of the second image;
a distance acquisition unit that acquires distance information indicating a distance between a subject of the medical observation device and the medical observation device; and
an arm control unit that controls the arm unit based on the light amount distributions of the first and second images and the distance information.

2. The medical arm control system according to claim 1, wherein the light amount acquisition unit acquires the light amount distribution of the second image from the light amount distribution of the first image.

3. The medical arm control system according to claim 1, further comprising
a sensor control unit that controls a gain of an image sensor of the medical observation device based on the light amount distributions of the first and second images and the distance information.

4. The medical arm control system according to claim 1, wherein an angle of view of the second image is narrower than an angle of view of the first image.

5. The medical arm control system according to claim 1, wherein the medical observation device is a wide-angle endoscope.

6. The medical arm control system according to claim 1, wherein
the medical observation device is a stereo endoscope, and
the distance acquisition unit acquires the distance information based on two of the first images by the stereo endoscope.

7. The medical arm control system according to claim 1, further comprising
a distance measuring device.

8. The medical arm control system according to claim 7, wherein the distance measuring device performs distance measurement using a ToF method or a structured light method.

9. The medical arm control system according to claim 1, further comprising a correction unit that corrects distortion of the first image.

10. The medical arm control system according to claim 1, further comprising a focus adjustment unit that automatically adjusts a focus of the medical observation device.

11. The medical arm control system according to claim 1, further comprising a magnification adjustment unit that automatically adjusts a magnification of the second image.

12. The medical arm control system according to claim 1, further comprising
an attitude recognition unit that recognizes an attitude of the arm unit.

13. The medical arm control system according to claim 12, wherein
the attitude recognition unit recognizes an attitude of the arm unit based on the first image.

14. The medical arm control system according to claim 12, wherein the attitude recognition unit recognizes the attitude of the arm unit based on sensing data from an inertial measurement device provided in the arm unit or lengths and angles of a plurality of elements included in the arm unit.

15. The medical arm control system according to claim 12, further comprising an instrument recognition unit that recognizes a medical instrument based on the first image.

16. The medical arm control system according to claim 15, wherein the range setting unit sets the cutout range for cutting out the second image based on at least one information of the distance information, the attitude of the arm unit, or the medical instrument.

17. The medical arm control system according to claim 1, further comprising the arm unit.

18. A medical arm control method, by a medical arm control device, comprising:
setting a cutout range in which a second image is cut out from a first image by a medical observation device supported by an arm unit;
acquiring a light amount distribution of the first image and a light amount distribution of the second image;
acquiring distance information indicating a distance between a subject of the medical observation device and the medical observation device; and
controlling the arm unit based on the light amount distributions of the first and second images and the distance information.

19. A program causing a computer to function as:
a range setting unit that sets a cutout range in which a second image is cut out from a first image by a medical observation device supported by an arm unit;
a light amount acquisition unit that acquires a light amount distribution of the first image and a light amount distribution of the second image;
a distance acquisition unit that acquires distance information indicating a distance between a subject of the medical observation device and the medical observation device; and
an arm control unit that controls the arm unit based on the light amount distributions of the first and second images and the distance information.
